(19) United States
(12) Patent Application Publication
(10) Pub. No.: US 2016/0033488 A1
(43) Pub. Date: Feb. 4, 2016

(54) Assay Enhancement by Selective Deposition and Binding on Amplification Structures

(71) Applicant: THE TRUSTEES OF PRINCETON UNIVERSITY, Princeton, NJ (US)

(72) Inventor: Stephen Y. CHOU, Princeton, NJ (US)

(21) Appl. No.: 14/775,634

(22) PCT Filed: Mar. 15, 2014
(86) PCT No.: PCT/US2014/029979

§ 371 Date: Sep. 11, 2015

Related U.S. Application Data
(60) Provisional application No. 61/801,424, filed on Mar. 15, 2013.

(51) Int. Cl. G01N 33/53; G01N 1/28

(52) U.S. Cl. CPC G01N 33/5306; G01N 1/2813

(57) ABSTRACT

This disclosure provides, among other things, a method for enhancing detection of an analyte that is bound to a substrate comprising a signal amplification layer on a surface of the substrate, wherein the signal amplification layer comprises high-amplification regions and low-amplification regions, and the high-amplification regions amplify signals at said surface more than the low-amplification regions. The method comprises selectively masking the low-amplification regions of the substrate, thereby increasing the probability that an analyte will bind to a high-amplification region and be detected.

### CROSS-REFERENCING

This application is a continuation-in-part of U.S. application Ser. No. 13/838,600, filed Mar. 15, 2013 (NSNR-003), which application claims the benefit of U.S. provisional application Ser. No. 61/622,226 filed on Apr. 10, 2012, and is a continuation-in-part of U.S. patent application Ser. No. 13/699,270, filed on Jun. 13, 2013, which application is a §371 filing of US2011/037455, filed on May 20, 2011, and claims the benefit of U.S. provisional application Ser. No. 61/347,178, filed on May 21, 2010;

This application is also a continuation-in-part of U.S. application Ser. No. 13/699,270, filed June 13, 2013 (NSNR-001), which application is a §371 filing of international application serial no. US2011/037455, filed on May 20, 2011, which application claims the benefit of U.S. Provisional Patent Application Ser. No. 61/347,178 filed on May 21, 2010; and

This application is also claims the benefit of: provisional application Ser. No. 61/801,424, filed Mar. 15, 2013 (NSNR-004PRV), provisional application Ser. No. 61/801,096, filed Mar. 15, 2013 (NSNR-005PRV), provisional application Ser. No. 61/800,915, filed Mar. 15, 2013 (NSNR-006PRV), provisional application Ser. No: 61/793,092, filed Mar. 15, 2013 (NSNR-008PRV), provisional Application Ser. No. 61/801,933, filed Mar. 15, 2013 (NSNR-009PRV), provisional Application Ser. No. 61/794,317, filed Mar. 15, 2013 (NSNR-010PRV), provisional application Ser. No.: 61/802,020, filed Mar. 15, 2013 (NSNR-011PRV) and provisional application SEr. No.: 61/802,223, filed Mar. 15, 2013 (NSNR-012PRV), all of which applications are incorporated by reference herein for all purposes.

### STATEMENT REGARDING FEDERALLY SPONSORED RESEARCH

This invention was made with government support under Grant No. FA9550-08-1-0222 awarded by the Defense Advanced Research Project Agency (DARPA). The government has certain rights in the invention.

### BACKGROUND

The invention is related to the methods, devices, fabrications and applications that can improve the property of assay sensing an analyte by selectively masking the surface and selectively bonding in an assay which has high sensing signal amplification surfaces and low sensing signal amplification surfaces.

The sensing includes Raman scattering, chromaticity, luminescence that includes fluorescence, electroluminescence, chemiluminescence, and electrochemiluminescence. The sensing property includes the sensing signal intensity, sensing signal spectrum, limit of detection, detection dynamic range, and signal variation reduction (smaller error bar) of the sensing. The invention can be used in the sensing in vitro, or in vivo.

To improve an assay sensing property, often an assay has an sensing signal amplification layer with micro/nanostructures on the surface of solid state support (e.g. plate), where the amplification layer has the areas that are high amplification surface and the rest area the low amplification area. One example of such amplification layer is a nanostructures plasmonic layer, such as D2PA.

The difference in analyte detection sensitivity between the high and low amplification areas can be a factor of 10 or larger. Often the high amplification areas are much smaller than that of the low amplification areas. Thus if the analytes bond to only the high amplification areas not in the low amplification areas, the sensing signal and related properties will be greatly enhanced over the case that the analyte bond to both high and low amplification area (or surface). Hence, there is a need to selectively mask the low amplification area to prevent analyte bonding in that area.

### BRIEF SUMMARY

The following brief summary is not intended to include all features and aspects of the present invention, nor does it imply that the invention must include all features and aspects discussed in this summary.

The invention is related to the methods, devices, fabrications and applications that can improve the property of assay sensing an analyte by selectively masking the surface and selectively bonding in an assay which has high sensing signal amplification surfaces and low sensing signal amplification surfaces. The sensing includes Raman scattering, chromaticity, luminescence that includes fluorescence, electroluminescence, chemiluminescence, and electrochemiluminescence. The sensing property includes the sensing signal intensity, sensing signal spectrum, limit of detection, detection dynamic range, and signal variation reduction (smaller error bar) of the sensing. The invention can be used in the sensing in vitro, or in vivo.

### BRIEF DESCRIPTION OF THE DRAWINGS

The skilled artisan will understand that the drawings, described below, are for illustration purposes only. The drawings are not intended to limit the scope of the present teachings in any way. Some of the drawings are not in scale.

FIG. 1 (a) Top view of a plate with a signal amplification layer, D2PA. (b) Cross-section of D2PA before shadow depositing masking material. (c) Masking material is shadow deposited from top downward in a direction normal to the plate surface; the deposited masking material covers the top surfaces of the metallic disks and the metallic backplane, but not the sidewall of the metallic structures not the metallic nanodots on the pillar sidewall.

FIG. 2 (a) After the deposition of masking materials, which mask a part of the metal (Au). (b) Coating molecular linkers that cover only metal, Au; (c) Bonding capture agents (e.g. antibody) only the molecular linkers.

FIG. 3 illustrates multiple (double) deposition of masking material at different angles for D2PA. (a) After the deposition of the first masking material shadow deposition. (b) The second masking material shadow deposition is deposited from a different angle to mask an area that was not masked by the first masking material. More than two masking material deposition can be used for masking designed area.

FIG. 4 illustrates other embodiments for the SAL layers: the disks on pillar (DoP).

FIG. 5 (a) SEM (scanning electron micrograph) of D2PA without coating. (b) The protein assay for the testing. And (c) Illustration that the capture agents and the analytes in the masked D2PA are bond to the high amplification area of the SAL, rather than all areas of the SAL as that in a unmasked D2PA. Other embodiments for the SAL layers include: the disks on pillar (DoP). shows an example of how directional evaporation of materials improve enhancement. By shadowing a cover layer on top of random metallic islands, IgG can only bond on the edge of metallic islands, where the highest field enhancement locates.

FIG. 6 Giant fluorescence enhancement was observed. The fluorescence enhancement in single masked D2PA is about 100 time better than the unmasked D2PA, and the double masked D2PA has an enhancement about 1.2-folds higher than the sing masked D2PA, compared to regular D2PA without masking.

FIG. 7 is a graph showing he limit of detection (LoD) of a masked D2PA is 0.9 aM, which is about 50 fold more sensitive than a normal D2PA with LoD of 43 aM.

Corresponding reference numerals indicate corresponding parts throughout the several figures of the drawings. It is to be understood that the drawings are for illustrating the concepts set forth in the present disclosure and are not to scale.

Before any embodiments of the invention are explained in detail, it is to be understood that the invention is not limited in its application to the details of construction and the arrangement of components set forth in the following description or illustrated in the drawings.

### DEFINITIONS

Before describing exemplary embodiments in greater detail, the following definitions are set forth to illustrate and define the meaning and scope of the terms used in the description.

The terms “disk-coupled dots-on-pillar antenna array” and “D2PA” as used herein refer to the device illustrated in in FIGS. 12 and 13, where the array 100 comprises: (a) substrate 110; and (b) a D2PA structure, on the surface of the substrate, comprising one or a plurality of pillars 115 extending from a surface of the substrate, wherein at least one of the pillars comprises a pillar body 120, metallic disc 130 on top of the pillar, metallic backplane 150 at the foot of the pillar, the metallic back plane covering a substantial portion of the substrate surface near the foot of the pillar; metallic dot structure 140 disposed on sidewall of the pillar. The D2PA amplifies a light signal that is proximal to the surface of the D2PA. The D2PA enhances local electric field and local electric field gradient in regions that is proximal to the surface of the D2PA. The light signal includes light scattering, light diffraction, light absorption, nonlinear light generation and absorption, Raman scattering, chromaticity, luminescence that includes fluorescence, electroluminescence, chemiluminescence, and electrochemiluminescence.

A D2PA array may also comprise a molecular adhesion layer that covers at least a part of said metallic dot structure, said metal disc, and/or said metallic back plane and, optionally, a capture agent that specifically binds to an analyte, wherein said capture agent is linked to the molecular adhesion layer of the D2PA array. The nanosensor can amplify a light signal from an analyte, when said analyte is bound to the capture agent. One preferred SAL embodiment is that the dimension of one, several or all critical metallic and dielectric components of SAL are less than the wavelength of the light in sensing. Details of the physical structure of disk-coupled dots-on-pillar antenna arrays, methods for their fabrication, methods for linking capture agents to disk-coupled dots-on-pillar antenna arrays and methods of using disk-coupled dots-on-pillar antenna arrays to detect analytes are described in a variety of publications including WO2012024006, WO2013154770, Li et al (Optics Express 2011 19, 3925-3936), Zhang et al (Nanotechnology 2012 23: 225-301); and Zhou et al (Anal. Chem. 2012 84: 4489) which are incorporated by reference for those disclosures.

The term “molecular adhesion layer” refers to a layer or multilayer of molecules of defined thickness that comprises an inner surface that is attached to the nanodevice and an outer (exterior) surface can be bound to capture agents.

The term “capture agent-reactive group” refers to a moiety of chemical function in a molecule that is reactive with capture agents, i.e., can react with a moiety (e.g., a hydroxyl, sulfhydryl, carboxy or amine group) in a capture agent to produce a stable strong, e.g., covalent bond.

The term “capture agent” as used herein refers to an agent that binds to a target analyte through an interaction that is sufficient to permit the agent to bind and concentrate the target molecule from a heterogeneous mixture of different molecules. The binding interaction is typically mediated by an affinity region of the capture agent. Typical capture agents include any moiety that can specifically bind to a target analyte. Certain capture agents specifically bind a target molecule with a dissociation constant (K_{D}) of less than about 10⁻⁶ M (e.g., less than about 10⁻⁷M, less than about 10⁻⁸M, less than about 10⁻⁹M, less than about 10⁻¹⁹ M, less than about 10⁻¹¹M, less than about 10⁻¹² M, to as low as 10⁻¹⁶ M) without significantly binding to other molecules. Exemplary capture agents include proteins (e.g., antibodies), and nucleic acids (e.g., oligonucleotides, DNA, RNA including aptamers).

The terms “specific binding” and “selective binding” refer to the ability of a capture agent to preferentially bind to a particular target molecule that is present in a heterogeneous mixture of different target molecule. A specific or selective binding interaction will discriminate between desirable (e.g., active) and undesirable (e.g., inactive) target molecules in a sample, typically more than about 10 to 100-fold or more (e.g., more than about 1000- or 10,000-fold).

The term “protein” refers to a polymeric form of amino acids of any length, i.e. greater than 2 amino acids, greater than about 5 amino acids, greater than about 10 amino acids, greater than about 20 amino acids, greater than about 50 amino acids, greater than about 100 amino acids, greater than about 200 amino acids, greater than about 500 amino acids, greater than about 1000 amino acids, greater than about 2000 amino acids, usually not greater than about 10,000 amino acids, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like. Also included by these terms are polypeptides that are post-translationally modified in a cell, e.g., glycosylated, cleaved, secreted, prenylated, carboxylated, phosphorylated, etc., and polypeptides with secondary or tertiary structure, and polypeptides that are strongly bound, e.g., covalently or non-covalently, to other moieties, e.g., other polypeptides, atoms, cofactors, etc.

The term “antibody” is intended to refer to an immunoglobulin or any fragment thereof, including single chain antibodies that are capable of antigen binding and phage display antibodies).

The term “nucleic acid” and “polynucleotide” are used interchangeably herein to describe a polymer of any length composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The term “complementary” as used herein refers to a nucleotide sequence that base-pairs by hydrogen bonds to a target nucleic acid of interest. In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. Typically, “complementary” refers to a nucleotide sequence that is fully complementary to a target of interest such that every nucleotide in the sequence is complementary to every nucleotide in the target nucleic acid in the corresponding positions. When a nucleotide sequence is not fully complementary (100% complementary) to a non-target sequence but still may base pair to the non-target sequence due to complementarity of certain stretches of nucleotide sequence to the non-target sequence, percent complementarily may be calculated to assess the possibility of a non-specific (off-target) binding. In general, a complementary of 50% or less does not lead to non-specific binding. In addition, a complementary of 70% or less may not lead to non-specific binding under stringent hybridization conditions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 200 nucleotides and up to 300 nucleotides in length, or longer, e.g., up to 500 nt in length or longer. Oligonucleotides may be synthetic and, in certain embodiments, are less than 300 nucleotides in length.

The term “attaching” as used herein refers to the strong, e.g, covalent or non-covalent, bond joining of one molecule to another.

The term “surface attached” as used herein refers to a molecule that is strongly attached to a surface.

The term “sample” as used herein relates to a material or mixture of materials containing one or more analytes of interest. In particular embodiments, the sample may be obtained from a biological sample such as cells, tissues, bodily fluids, and stool. Bodily fluids of interest include but are not limited to, amniotic fluid, aqueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine and exhaled condensate. In particular embodiments, a sample may be obtained from a subject, e.g., a human, and it may be processed prior to use in the subject assay. For example, prior to analysis, the protein/nucleic acid may be extracted from a tissue sample prior to use, methods for which are known. In particular embodiments, the sample may be a clinical sample, e.g., a sample collected from a patient.

The term “analyte” refers to a molecule (e.g., a protein, nucleic acid, or other molecule) that can be bound by a capture agent and detected.

The term “assaying” refers to testing a sample to detect the presence and/or abundance of an analyte.

As used herein, the terms “determining,” “measuring,” and “assessing,” and “assaying” are used interchangeably and include both quantitative and qualitative determinations.

As used herein, the term “light-emitting label” refers to a label that can emit light when under an external excitation. This can be luminescence. Fluorescent labels (which include dye molecules or quantum dots), and luminescent labels (e.g., electro- or chemi-luminescent labels) are types of light-emitting label. The external excitation is light (photons) for fluorescence, electrical current for electroluminescence and chemical reaction for chemi-luminscence. An external excitation can be a combination of the above.

The phrase “labeled analyte” refers to an analyte that is detectably labeled with a light emitting label such that the analyte can be detected by assessing the presence of the label. A labeled analyte may be labeled directly (i.e., the analyte itself may be directly conjugated to a label, e.g., via a strong bond, e.g., a covalent or non-covalent bond), or a labeled analyte may be labeled indirectly (i.e., the analyte is bound by a secondary capture agent that is directly labeled).

The term “hybridization” refers to the specific binding of a nucleic acid to a complementary nucleic acid via Watson-Crick base pairing. Accordingly, the term “in situ hybridization” refers to specific binding of a nucleic acid to a metaphase or interphase chromosome.

The terms “hybridizing” and “binding”, with respect to nucleic acids, are used interchangeably.

The term “capture agent/analyte complex” is a complex that results from the specific binding of a capture agent with an analyte. A capture agent and an analyte for the capture agent will usually specifically bind to each other under “specific binding conditions” or “conditions suitable for specific binding”, where such conditions are those conditions (in terms of salt concentration, pH, detergent, protein concentration, temperature, etc.) which allow for binding to occur between capture agents and analytes to bind in solution. Such conditions, particularly with respect to antibodies and their antigens and nucleic acid hybridization are well known in the art (see, e.g., Harlow and Lane (Antibodies: A Laboratory Manual Cold Spring Harbor Laboratory, Cold Spring Harbor, N.Y. (1989) and Ausubel, et al, Short Protocols in Molecular Biology, 5th ed., Wiley & Sons, 2002).

The term “specific binding conditions” as used herein refers to conditions that produce nucleic acid duplexes or protein/protein (e.g., antibody/antigen) complexes that contain pairs of molecules that specifically bind to one another, while, at the same time, disfavor to the formation of complexes between molecules that do not specifically bind to one another. Specific binding conditions are the summation or combination (totality) of both hybridization and wash conditions, and may include a wash and blocking steps, if necessary.

For nucleic acid hybridization, specific binding conditions can be achieved by incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C.

For binding of an antibody to an antigen, specific binding conditions can be achieved by blocking a substrate containing antibodies in blocking solution (e.g., PBS with 3% BSA or non-fat milk), followed by incubation with a sample containing analytes in diluted blocking buffer. After this incubation, the substrate is washed in washing solution (e.g. PBS+TWEEN 20) and incubated with a secondary capture antibody (detection antibody, which recognizes a second site in the antigen). The secondary capture antibody may conjugated with an optical detectable label, e.g., a fluorophore such as IRDye800CW, Alexa 790, Dylight 800. After another wash, the presence of the bound secondary capture antibody may be detected. One of skill in the art would be knowledgeable as to the parameters that can be modified to increase the signal detected and to reduce the background noise.

The term “a secondary capture agent” which can also be referred to as a “detection agent” refers a group of biomolecules or chemical compounds that have highly specific affinity to the antigen. The secondary capture agent can be strongly linked to an optical detectable label, e.g., enzyme, fluorescence label, or can itself be detected by another detection agent that is linked to an optical detectable label through bioconjugatio (Hermanson, “Bioconjugate Techniques” Academic Press, 2nd Ed., 2008).

The term “biotin moiety” refers to an affinity agent that includes biotin or a biotin analogue such as desthiobiotin, oxybiotin, 2′-iminobiotin, diaminobiotin, biotin sulfoxide, biocytin, etc. Biotin moieties bind to streptavidin with an affinity of at least 10-8M. A biotin affinity agent may also include a linker, e.g., -LC-biotin, -LC-LC-Biotin, -SLC-Biotin or -PEGn-Biotin where n is 3-12.

The term “streptavidin” refers to both streptavidin and avidin, as well as any variants thereof that bind to biotin with high affinity.

The term “marker” refers to an analyte whose presence or abundance in a biological sample is correlated with a disease or condition.

The term “bond” includes covalent and non-covalent bonds, including hydrogen bonds, ionic bonds and bonds produced by van der Waal forces.

The term “amplify” refers to an increase in the magnitude of a signal, e.g., at least a 10-fold increase, at least a 100-fold increase at least a 1,000-fold increase, at least a 10,000-fold increase, or at least a 100,000-fold increase in a signal.

Other specific binding conditions are known in the art and may also be employed herein.

It must be noted that as used herein and in the appended claims, the singular forms “a”, “an”, and “the” include plural referents unless the context clearly dictates otherwise, e.g., when the word “single” is used. For example, reference to “an analyte” includes a single analyte and multiple analytes, reference to “a capture agent” includes a single capture agent and multiple capture agents, and reference to “a detection agent” includes a single detection agent and multiple detection agents.

### DETAILED DESCRIPTION OF EXEMPLARY EMBODIMENTS

The following detailed description illustrates some embodiments of the invention by way of example and not by way of limitation.

The invention is related to the methods and devices that can improve the property of an assay in sensing an analyte, and their making and use. The invention is related to the assays that have a signal amplification surface that captures the analytes and has high signal amplification areas and low-amplification areas. The invention is related to the methods to selectively mask the low signal amplification areas, so that the analytes will be bond to the high signal amplification area, therefore improve the sensing property.

The analyte include proteins, peptides, DNA, RNA, nucleic acid, small molecules, cells, nanoparticles with different shapes. The targeted analyte can be either in a solution or in air or gas phase. The sensing includes light absorption, light scattering, light radiation, Raman scattering, chromaticity, luminescence that includes fluorescence, electroluminescence, chemiluminescence, and electrochemiluminescence. The sensing property includes the sensing signal intensity, sensing signal spectrum, limit of detection, detection dynamic range, and signal variation reduction (smaller error bar) of the sensing. The invention can be used in the sensing in vitro, or in vivo. The assay with a signal amplification layer is sometimes termed as “nanosensor” because of their nanostructures.

To improve an assay sensing property, often an assay has a sensing signal amplification (SAL) layer on the surface of solid state support (e.g. plate), where the capture agents are attached, which in turn capture the analyte. The SAL layer often comprises with micro/nanostructures of metallic and dielectric materials. Within the surface of the SAL where capture agents to be attached, often it further divides into the areas of high signal amplification and the other area of low signal amplification. The difference in signal amplification between the high and low amplification areas can be a factor of 10 or larger.

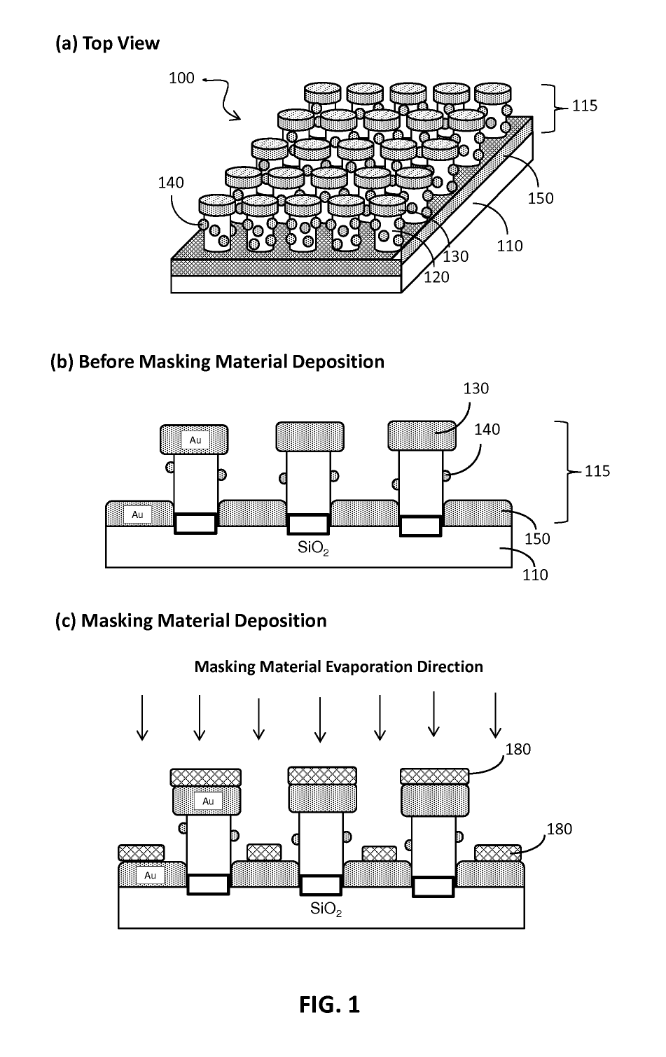

One example of such signal amplification layer is a nanostructures plasmonic layer in D2PA (disk-coupled dots-on-pillar antenna array) (FIG. 1). The high amplification areas are the areas with sharp (i.e. small curvature) edges of metallic materials and the small gaps between two metallic materials.

Furthermore, often for a given signal amplification surface, the high amplification areas are much smaller than the low amplification areas. Thus if the analytes bond to only the high amplification areas but not the low amplification areas, the signal sensing sensitivity and other related sensing properties will be greatly enhanced, compared to the situation that the analytes have the same probability to bind the high and low amplification area (or surface).

The invention is related to the methods that make the analytes bind to the high amplification areas only but not to the low amplification area. The methods comprise selectively modification of at least a portion of the low amplification areas while keep at least a portion of the high amplification area unmodified, so that the capture agents have a higher probability bonding to the high amplification area than to the low amplification area.

The modification can be several ways, including (a) depositing a masking material to the low amplification areas that reduce the capture agents bonding, (b) depositing an adhesion material to the high amplification areas that increases the capture agents bonding, (c) changing the surface (one or a few atomic layer) chemical properties of the low amplification areas that reduce the capture agents bonding, and (e) changing the surface (one or a few atomic layer) chemical properties of the high amplification areas that increase the capture agents bonding. The many case the modified low amplification area does not bond the capture agent or the bonding is so weak that a simple watch will remove them. For an assay to work properly, the unspecific bonding of the analytes on the assay surface should be very low.

And (2) using the capture agents that do not bond to the masking material but the surface material(s) in the uncovered (i.e. unmasked) high amplification area. In some cases in step (2), a molecular adhesion layer is used to link between the high amplification area and the capture agents. For example, in D2PA, a molecular adhesion layer is a SAM layer dithiobis(succinimidyl undecanoate) (DSU). The DSU SAM layer binds to SAL's metal surface through sulfer-gold bond, and has a terminal group of NHS-ester that binds to the primary amine sites on many protein capture agents.

The selective deposition to mask the low amplification area has several ways. One way is to selectively deposit the masking material on the low amplification area only. Another way is to deposit a masking material everywhere (i.e. both the low and high amplification areas) and then either selectively remove the masking materials from the high amplification area, or selectively deposit another materials on the high amplification area that can attach the capture agents.

The selective deposition of the masking materials can be achieved in several ways, including (a) using lithography, deposition and lift-off, (b) using shadow deposition, (d) deposition using a shadow mask, and (d) using others. The shadow deposition utilizes a 3D (three-dimensional) surface topology of a signal amplification layer to selectively over a portion of the surface. In the deposition using a shadow mask, the masking material is deposited on the selected area of the SAL layer though a shadow mask. A shadow mask is a plate with holes that can let materials or energetic beam through, while blocking the materials in other area.

An alternative to material deposition is to use a directional energetic beam (photons, electrons, ions and alike) to modify the exposed surface chemistry, so that a functional head group of molecules will bond the modified surface but not to unmodified surface, or vice versa. The modification can be in environment of a gas.

For example, one can oxidize the surface a metal or semiconductors (e.g. silicon) by shining an energetic beam in an oxygen gas environment.

One embodiment of the invention is the method of improving the property (including the sensitivity) of an assay of sensing an analyte by selectively masking (i.e. blocking) the low signal amplification area while leaving the high signal amplification area open for catching the analyte.

One embodiment of the invention is the method of selective masking of low amplification area is by shadow deposition of the masking materials that the capture agents would not bond.

One embodiment of the invention is the method to achieve selective masking that use the capture agent with an end function group that bond the materials in the high amplification area but not the material in the low amplification area.

One embodiment of the invention is that the masking materials are deposited multiple times either from the same deposition angle or different angles for achieving the purpose of higher sensing signal.

The invention can be used for improving different sizes of assays from 1 micrometer to 100 centimeter or larger. It also can be used for assays inside a microfluidic channel.

### Sensing Amplification Surface

The methods of the invention applies to any assays that have a SAL layer that have has high amplification and low amplification regions. In many of assays, the sensing amplification surface has micro/nanostructures of metallic (plasmonic) and dielectric materials. The high signal amplification regions are the regions that have sharp curvatures and/or between a small gap of two metallic structures. Some exemplary such assay embodiments are the follows.

One of the assay is the D2PA assay, as described in the Definition. In the D2PA, the high signal amplification regions are around the metallic nano-dots, the edges of the metallic disks, and the edges of the metallic backplane, and between the small gaps between all metallic parts. The low sensing amplification regions are the top surface of the metallic disk and metallic backplane. Clearly, the total areas of the high amplification area are much smaller than that of the low amplification area. In a D2PA without a selective masking, the capture agents will be attached rather uniformly, either over all metallic surfaces or all open surfaces, depending upon the bonding chemistry, thus having only small fraction of the analytes captured at the high amplification area.

One preferred D2PA operating for light signal in ˜800 nm wavelength comprises a periodic non-metallic (e.g. dielectric or semiconductor) pillar array (200 nm pitch and ˜100 nm diameter), a metallic disk (˜135 nm diameter) on top of each pillar, a metallic backplane on the foot of the pillars, metallic nanodots randomly located on the pillar walls, and nanogaps between these metal components. The nanodots have diameters of ˜5-20 nm, and the nanogaps between them and the nanodisks are 1-10 nm. The disks have a diameter slightly larger than the pillar, hence having an overhang.

Another embodiment of the sensing implication surface comprises a or a plural of metallic discs and a significantly continuous metallic film, wherein the significant part of the metallic disc has a separation from the metallic film. The separation is 0.5 to 30 nm, and the average disc's lateral dimension is from 20 nm to 250 nm.

Another embodiment of the sensing implication surface comprises a or a plural of metallic discs on a substrate and the average disc's lateral dimension of from 20 nm to 250 nm, and has at least a gap of 0.5 to 30 nm between the two adjacent discs.

The metallic disk in all embodiments has a shape selected from the group of shapes consisting of round, polygonal, pyramidal, elliptical, elongated bar shaped, or any combination thereof.

The metal may be gold, silver, platinum, palladium, lead, iron, titanium, nickel, copper, aluminum, alloy thereof, or combinations thereof, although other materials may be used, as long as the materials' plasma frequency is higher than that of the light signal and the light that is used to generate the light signal.

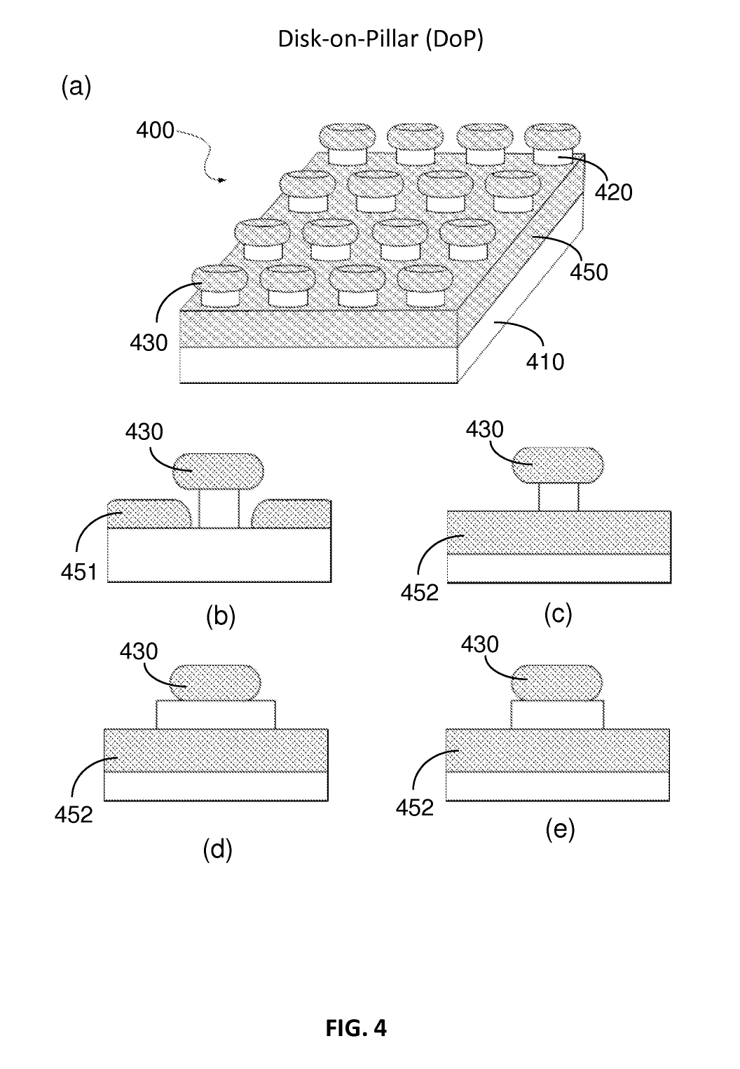

Other embodiments for the SAL layers are the disks on pillar (DoP) 400, shown in FIG. 4, that comprise a substrate 410; substantially continuous metallic film 420, one or a plurality of pillars extending from a surface of the substrate, wherein at least one of the pillars comprises a pillar body 420, metallic disc 430 on top of the pillar, and metallic backplane 450. The metallic back plane can be either type A 451: at the foot of the pillar covering a substantial portion of the substrate surface near the foot of the pillar; or type B 452: a sheet of film go under the pillar. The discs can have a lateral dimension either larger (preferred) or smaller or the same as the pillars.

For enhancing light of a wavelength of 400 nm to 1,000 nm (visible to near-infrared), the separation is 0.5 to 30 nm, the average disc's lateral dimension is from 20 nm to 250 nm, and the disk thickness is from 10 nm to 60 nm, depending upon the light wavelength used in sensing.

The size of the assay substrate can be array from large in 10's centimeters for in vitro applications to 1 micrometer for in vivo applications. When the substrate size is very small, they are usually fabricated on a large wafer first and then are cut into the small sized. The substrate can be any materials, but may be limited by chemical reactivity or plasmonic effects required by the signal amplification layers.

### Shadow Deposition for Selective masking methods.

The shadow deposition of a materials refers that the disposition where the material is deposited in the form of a beam from a given direction (FIG. 1c) toward a surface with a 3D topology. Just like a telephone pole blocks the Sun light having a shadow, some of the surface topological structure will block the material beam leaving a “shadow” behind, and hence no materials are deposited in the shadow area. The area to be deposited and to be masked in a shadow deposition is determined by the angle of the shadow deposition and by the surface topology. The masking materials can be any materials that do not bond the capture agents and the analytes. In many cases, the shadow deposition can be only partially directional.

### Angles for Shadow Deposition of Masking Materials

The angle of the shadow deposition to enhance an assay is determined by the position of the high and low amplification area. For the D2PA or the SAL layers with similar topology, the high amplification areas are mainly on the side of pillars, and the low amplification areas are the top of the pillar and the flat surface at the foot of the pillars. Therefore for the D2PA and alike, a shadow deposition of masking material 180 with an angle normal to the surface will mask the most of the low amplification area. As shown in FIG. 1, by a shadow deposition in a normal direction, the deposited masking material 180 sits on the top of each metallic disk 130, and the top of the metallic backplane 150, while leaving the metallic nanodots, the edges of the metallic disks and backplanes, and the nanogaps between metallic components unmasked. In one example, the masking material is silicon dioxide. The typical thickness is about 1 nm to 10 nm.

### Multiple Shadow Depositions of Masking Materials.

The masking materials can be deposited for multiple times from the same deposition angle or different angle for achieving the purpose of higher sensing signal. The deposition angle refers the angle between the deposition beam and the norm of the SAL surface.

The use of multiple deposition in different angles allows covering more areas that are needed to be covered. By choosing proper number of deposition and proper angle (or angles), one can have certain high amplification areas selected for the analytes bonding, while having all other areas masked to prevent a bonding (more or less). Another purpose of the selective masking is to precise control the bonding sites of the analytes. The position control has certain advantages in certain signal reading and analysis methods.

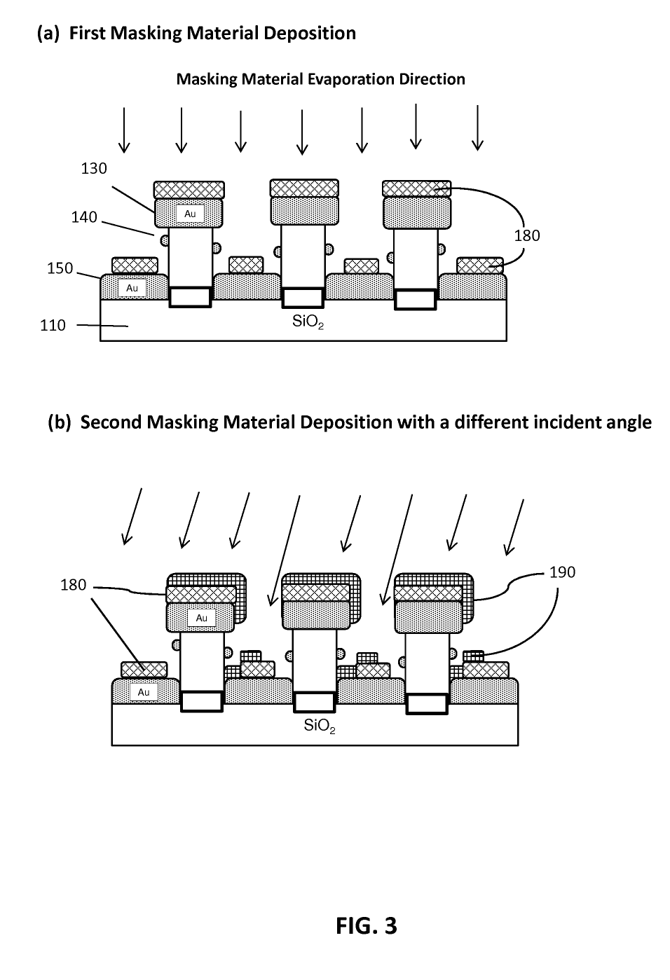

FIG. 3 illustrates a double shadow deposition from two angles for D2PA. The first shadow deposition covers the metallic disks 130 on top of the pillars and the metallic backplane 150 with a masking material 180, while leaving the metallic nanodots, the edges of the metallic disks and backplanes, and the nanogaps between metallic components unmasked. In the second shadow deposition, the masking material 190, covers a part of the edge of the disk and the backplane, thus making more capture agent bond to the nanodot 140, where the amplification are among the highest.

### Masking Materials and Thickness

The masking materials can be selected from that any materials that prevent the bonding or create a bonding of the capture agents (note they also should not have no or small nonspecific bonding of the analytes) The thickness can be from 0.1 nm to 200 nm as long as it function as the masking. Another consideration for selecting the masking material and thickness is the resonant wavelength of the amplification layer; they should not adversary affect significantly of the resonance which is the key for the amplification.

The masking materials can be dielectrics and semiconductors, and can be in the form of amorphous, crystals, polycrystalline, small molecules, large molecules, etc. One common masking materials is silicon dioxide. Another is polymers such as polystyrene, PMMA. Other suitable masking materials include silicon nitride and diblock copolymer composed of PS-b-PMMA, a PS-r-PMMA random copolymer (see, e.g., U.S. Pat. No. 8,513,359) and other amorphous dielectric materials includes. In certain cases, a copolymer may be selected from a group consisting of polystyrene-block-polymethylmethacrylate (PS-b-PMMA), polystyrene-block-polyisoprene (PS-b-PI), polystyrene-block-polybutadiene (PS-b-PBD), polystyrene-block-polyvinylpyridine (PS-b-PVP), polystyrene-block-polyethyleneoxide (PS-b-PEO), polystyrene-block-polyethylene (PS-b-PE), polystyrene-b-polyorganosilicate (PS-b-POS), polystyrene-block-polyferrocenyldimethylsilane (PS-b-PFS), polyethyleneoxide-block-polyisoprene (PEO-b-PI), polyethyleneoxide-block-polybutadiene (PEO-b-PBD), polyethyleneoxide-block-polymethylmethacrylate (PEO-b-PMMA), polyethyleneoxide-block-polyethylethylene (PEO-b-P EE), polybutadiene-block-polyvinylpyridine (PBD-b-PVP), and polyisoprene-block-polymethylmethacrylate (PI-b-PMMA).

The thickness of masking material is preferred to be adjusted for the best masking effects. For examples, in a real directional deposition, a small amount of masking material may stray away from the deposition direction and get into the shadow area. In this case, the thickness of the deposition should be reduced to make the stray away masking materials so minute, that it covers only small part of the shadowed area. The typical thickness of the masking material is 0.5 nm to 150 nm.

The examples of D2PA's structure, materials, surface functionalization (bonding chemistry), detections, and applications (e.g. biological/chemical detection and disease detections) have been described, which are ALL applicable to the current invention (see, e.g., Li et al Optics Express 2011 19, 3925-3936, WO2012/024006, and patent application entitled “Ultra-Sensitive Sensors” (included as a part of the description) which are incorporated by reference).

### Methods to Shadow Depositing Materials

The methods to shadow deposit materials can be any method, as long as it is more or less directional, and can evaporate the intended materials. The deposition methods include evaporation, sputtering and chemical or molecular beams. The evaporation further includes the evaporation by chemical vapors, molecular beams, electron beam heating thermal heating, laser heating, and other heating methods. The sputtering includes the sputtering by ion, electron, plasmon, photon (i.e. laser), and other energetic particles.

### Capture Agents and Molecular Adhesion Layers

The capture agents should be selectively bond to the selected high amplification area that are either unmasked or bonding (with the capture agents) enhanced.

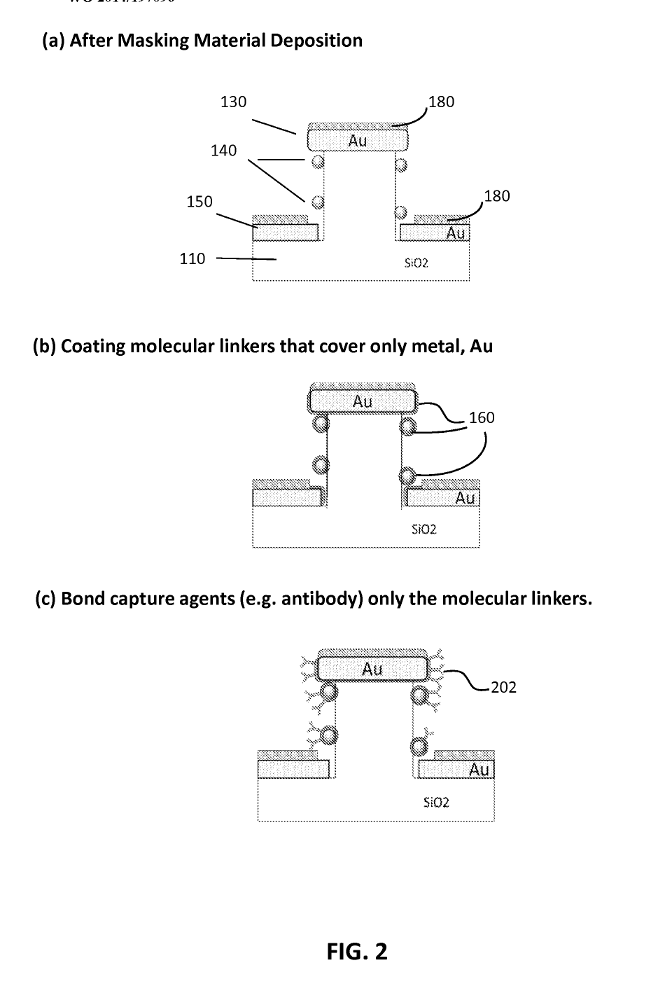

In one embodiment, the capture agent does not bond or weakly bond to all materials on the SAL layer surface, but a molecular adhesion layer (MAL) is used to link the capture agent to the desired surface. For example, in D2PA, the MAL 160 is selected coated in the gold, as shown in FIG. 2.

In one embodiment of the MAL for D2PA and alike, the molecular adhesion layer 160 is a self-assembled monolayer (SAM) of cross-link molecules or ligands, each molecule for the SAM comprises of three parts: (i) head group, which has a specific chemical affinity to the metal surface, (ii) terminal group, which has a specific affinity to the capture agent, and (iii) molecule chain, which is a long series of molecules that link the head group and terminal group, and its length (which determines the average spacing between the metal to the capture agent) can affect the light amplification of the assay.

As an example, the molecular adhesion layer, may contain a SAM layer dithiobis(succinimidyl undecanoate) (DSU). The DSU SAM layer binds to SAL's metal surface through sulfer-gold bond, and has a terminal group of NHS-ester that binds to the primary amine sites on many protein capture agents. One example is in D2PA where the capture agent 202 bond to the gold through the MAL 160.

The molecular adhesion layer can have many different configurations, including (a) a self-assembled monolayer (SAM) of cross-link molecules, (b) a multi-molecular layers thin film, (c) a combination of (a) and (b), and (d) a capture agent itself.

Various method for linking capture agents to a metal surface, with or without a molecular linking layer, are described in WO2013154770, which is incorporated by reference for such methods. For example, in some cases, the metal surface may be first joined to one end (e.g., via a thiol or silane head group) of a molecule of a defined length (e.g., of 0.5 nm to 50 nm in length) and the capture agent can be linked to the other end of the molecule via a capture agent-reactive group (e.g., an N-hydroxysuccinimidyl ester, maleimide, or iodoacetyl group). Dithiobis(succinimidyl undecanoate), which has a —SH head group that binds to a gold surface through sulfer-gold bond, and an NHS-ester terminal group that reacts with primary amines, may be used in certain cases.

### Assays

The analyte include proteins, peptides, DNA, RNA, nucleic acid, small molecules, cells, nanoparticles with different shapes. The targeted analyte can be either in a solution or in air or gas phase. The sensing includes light absorption, light scattering, light radiation, Raman scattering, chromaticity, luminescence that includes fluorescence, electroluminescence, chemiluminescence, and electrochemiluminescence. The sensing property includes the sensing signal intensity, sensing signal spectrum, limit of detection, detection dynamic range, and signal variation reduction (smaller error bar) of the sensing. The invention can be used in the sensing in vitro, or in vivo. The assay with a signal amplification layer is sometimes termed as “nanosensor” because of their nanostructures. Various assays and applications are described in WO2013154770, which is incorporated by reference for such methods.

In some assays, the biosensor is linked to an antibody in accordance with the methods described above to produce a biosensor comprises antibodies that are linked to the molecular adhesion layer of the biosensor. After the biosensor has been produced, the biosensor is contacted with a sample containing a target analyte (e.g., a target protein) under conditions suitable for specific binding. The antibodies specifically bind to target analyte in the sample. After unbound analytes have been washed from the biosensor, the biosensor is contacted with a secondary antibody that is labeled with a light-emitting label under conditions suitable for specific binding. After unbound secondary antibodies have been removed from the biosensor, the biosensor may be read to identify and/or quantify the amount of analyte in the initial sample.

In other assays, the biosensor is linked to a nucleic acid, e.g., an oligonucleotide in accordance with the methods described above to produce a biosensor that comprises nucleic acid molecules that are linked to the molecular adhesion layer. After the biosensor has been produced, the biosensor is contacted with a sample containing target nucleic acid under conditions suitable for specific hybridization of target nucleic acid to the nucleic acid capture agents. The nucleic acid capture agents specifically binds to target nucleic acid in the sample. After unbound nucleic acids have been washed from the biosensor, the biosensor is contacted with a secondary nucleic acid that is labeled with a light-emitting label under conditions for specific hybridization. After unbound secondary nucleic acids have been removed from the biosensor, the biosensor may be read to identify and/or quantify the amount of nucleic acid in the initial sample.

In these embodiments, bound analyte can be detected using a secondary capture agent (i.e. the “detection agent”) may be conjugated to a fluorophore or an enzyme that catalyzes the synthesis of a chromogenic compound that can be detected visually or using an imaging system. In one embodiment, horseradish peroxidase (HRP) may be used, which can convert chromogenic substrates (e.g., TMB, DAB, or ABTS) into colored products, or, alternatively, produce a luminescent product when chemiluminescent substrates are used. In particular embodiments, the light signal produced by the label has a wavelength that is in the range of 300 nm to 900 nm). In certain embodiments, the label may be electrochemiluminescent and, as such, a light signal can be produced by supplying a current to the sensor.

In some embodiments, the secondary capture agent (i.e. the detection agent), e.g., the secondary antibody or secondary nucleic acid, may be linked to a fluorophore. Methods for labeling proteins, e.g., secondary antibodies, and nucleic acids with fluorophores are well known in the art. Chemiluminescent labels include acridinium esters and sulfonamides, luminol and isoluminol; electrochemiluminescent labels include ruthenium (II) chelates, and others are known.

### Examples-1. D2PA with Single and Double Shadow Deposition

We have experimentally demonstrated the method of the subject invention. We used SiO2 as the masking materials and evaporated them directionally from top in a vertical direction to the surface of D2PA. The capture agents are bond to the uncovered gold only. The assay is enhanced by ˜50 to 100 times.

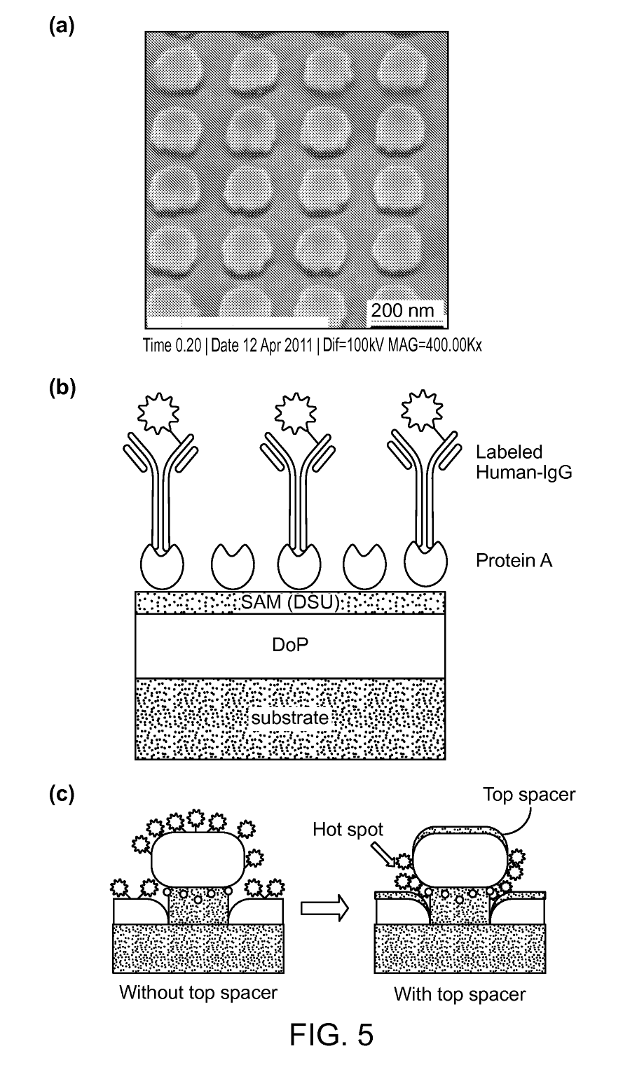

The fabrication process (a) SiO2 layer is thermally grown on silicon; (b) nanoimprint is performed by using a 200 nm-pitch pillar mold; (c) after residual resist etching, Cr pads are evaporated and lift-off; (d) SiO2 layer is etched into pillar array masked by Cr pads. (e) 40 nm gold is evaporated to self-form D2PA structure. The SEM of D2PA without coating is shown in FIG. 5.a.

Single masking shadow deposition, (4 nm-SiO2 masking material is deposited for the normal direction. For the double shadow deposition, the wafer is tilted and the angle is 30°). The deposited SiO2 masking thickness is 3 nm.

As shown in FIG. 5b. elf-assemble DSU monolayer as the molecular adhesion layer. Use human IgG as the capture agent. Blocking with BSA. Add anti-IgG labeled with IRDye800CW as the detection agent. The capture agents and the analytes in the masked D2PA are bond to the high amplification area of the SAL, rather than all areas of the SAL as that in a unmasked D2PA, as illustrated in FIG. 5c.

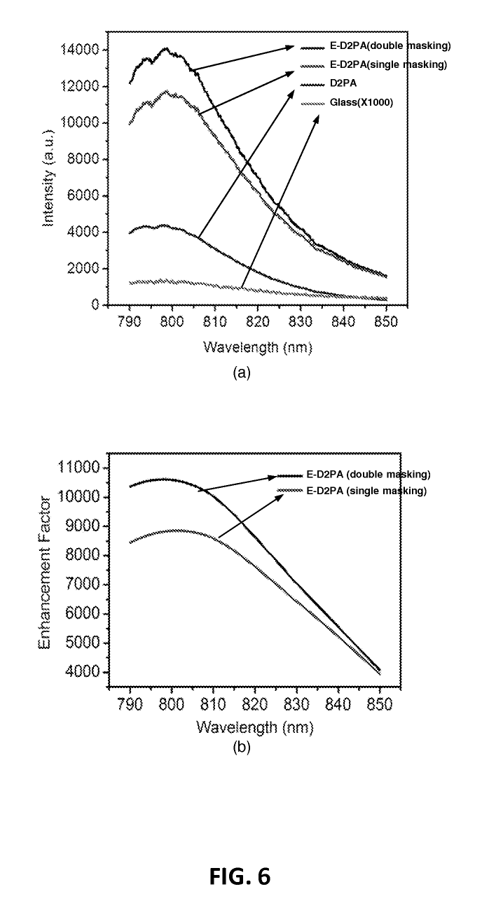

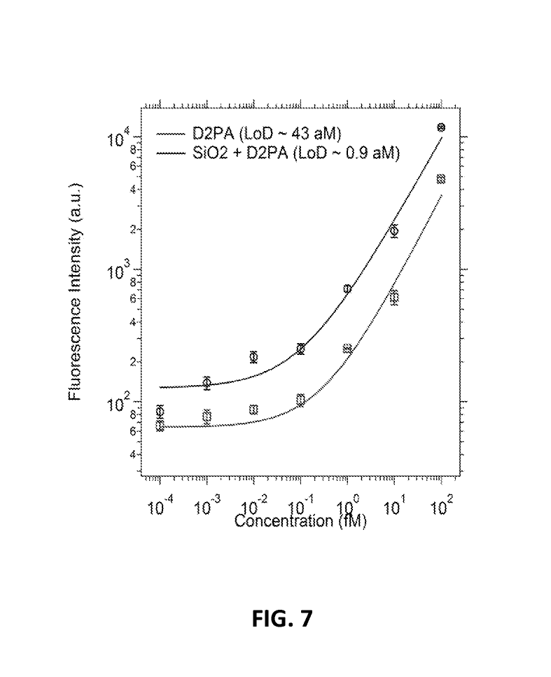

Giant fluorescence enhancement was observed (FIG. 6). The fluorescence enhancement in single masked D2PA is about 100 time better than unmasked D2PA, and the double masked D2PA has an enhancement about 1.2-folds higher than the sing masked D2PA. FIG. 7 a shows the limit of detection (LoD) of a single masked D2PA is 0.9 aM, which is about 50 fold more sensitive than a normal D2PA with LoD of 43 aM.

### Applications

The applications of the subject sensor include, but not limited to, (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

The detection can be carried out in various sample matrix, such as cells, tissues, bodily fluids, and stool. Bodily fluids of interest include but are not limited to, amniotic fluid, aqueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine and exhaled condensate.

Although the foregoing embodiments have been described in some detail by way of illustration and example for purposes of clarity of understanding, it is readily apparent to those of ordinary skill in the art in light of the above teachings that certain changes and modifications can be made thereto without departing from the spirit or scope of the appended claims.

What is claimed is:

1. A method for enhancing detection of an analyte that is bound to a substrate, comprising:
(a) obtaining a substrate comprising a signal amplification layer on a surface of the substrate, wherein the signal amplification layer comprises high-amplification areas and low-amplification areas, and wherein the high-amplification regions amplify signals at said surface more than the low-amplification regions;
(b) selectively modifying the low-amplification areas and/or the high amplification areas of the substrate, thereby increasing the probability of the binding of an analyte to a high-amplification region and/or reduce the probability of the binding of an analyte to a low-amplification area;
thereby improving the sensitivity of detecting said analyte and/or other sensing properties.

2. The method of claim 1, wherein the selectively modifying comprises depositing a masking material to the low amplification areas to reduce capture agent bonding.

3. The method of any prior claim, wherein the selectively modifying comprises depositing an adhesion material to the high amplification areas to increase capture agent bonding.

4. The method of any prior claim, wherein the selectively modifying comprises changing the surface chemical properties of the low amplification areas to reduce bonding of capture agents to the low amplification areas.

5. The method of any prior claim, wherein the selectively modifying comprises changing the surface chemical properties of the high amplification areas to increase bonding of capture agents to the high amplification areas.

6. The method of any prior claim, wherein the modification comprises a shadow deposition.

7. The method of any prior claim, wherein the modification comprises multiple shadow depositions from the same or multiple different deposition angles.

8. The method of any prior claim, wherein the selectively modifying is done by masking the low-amplification areas.

9. The method of claim 8, wherein the masking is done using PMMA, polystyrene, a co-block polymer, silicon dioxide or silicon nitride.

10. The method of any prior claim, wherein the method further comprises attaching capture agents to the high amplification areas, wherein the capture agents selectively bind the analytes.

11. The method of any prior claim, wherein the analyte is selected from the group consisting of a protein, peptide, DNA, RNA, nucleic acid, small molecule, cell, and a nanoparticle with different shapes.

12. The method of any prior claim, wherein the target analytes are labeled with a label, either prior to or after they are bound to said the high-amplification region.

13. The method of any prior claim, wherein the signal that is amplified is Raman scattering, chromaticity, luminescence, fluorescence, electroluminescence, chemiluminescence, and/or electrochemiluminescence.

14. The method of any prior claim, wherein the selective masking enhances one or more of signal intensity, sensing signal spectrum, limit of detection, detection dynamic range, and signal variation reduction (smaller error bar) of the sensing,

15. The method of any prior claim, wherein the detecting include (a) measuring a lump-sum of signal over an area, or (b) counting individual binding events in an area of the image, thereby providing an estimate of the amount of one or more analytes in the sample.

16. The method of any prior claim, wherein the signal amplification layer is a D2PA.

17. The method of any prior claim, wherein the signal amplification layer comprises one or a plural of metallic discs and a significantly continuous metallic film, wherein the significant part of the metallic disc is separated from the metallic film.

18. The method of claim 17, wherein the metallic disk has a shape selected from the group of shapes consisting of round, polygonal, pyramidal, elliptical, elongated bar shaped, or any combination thereof.

19. The method of claim 17, wherein the metallic disc is separated from the metallic film by a distance in the range of 0.5 to 30 nm, and the average lateral dimension of the discs is in the range of 20 nm to 250 nm.

20. The method of any prior claim, wherein the signal amplification layer comprises one or more metallic discs has a shape selected from the group of shapes consisting of round, polygonal, pyramidal, elliptical, elongated bar shaped, or any combination thereof, wherein the average lateral dimension of the discs is in the range 20 nm to 250 nm, and the gap between adjacent discs in the range of 0.5 to 30 nm.

21. The method of any prior claim, wherein the high amplification region include the surface.

22. The method of any prior claim, wherein the high amplification region are the region with metallic nanostructures of sharp curvature, or the regions of a small gap between to metallic structures.

23. The method of any prior claim, wherein the selective masking comprise deposition of a masking material, more or less, in the form of a beam from one direction toward the amplification surface.

24. The method of claim 23, wherein the direction has a different angle with the amplification surface, depending on the masking areas.

25. The method of claim 23, wherein the directional deposition can be multiple depositions at different angles.

26. The method of any prior claim, wherein the masking material is PMMA, polystyrene, a co-block polymer, silicon dioxide or silicon nitride

27. The method of any prior claim, wherein the mask is of a thickness of 1 nm to 10 nm.

28. The method of any prior claim, wherein the signal amplification layer is inside a microfluidic channel.

29. A sensing substrate comprising a signal amplification layer on a surface, wherein the signal amplification layer comprises high-amplification regions and low-amplification regions, wherein the high-amplification regions amplify signals at said surface more than the low-amplification regions, wherein the low-amplification regions of the substrate have been selectively masked, thereby increasing the probability that an analyte will bind to a high-amplification region and be detected.

30. The sensing substrate of claim 29, wherein the masking material is PMMA, polystyrene, a co-block polymer, silicon dioxide or silicon nitride

31. The sensing substrate of claim 29 or 30, wherein the mask is of a thickness of 1 nm to 10 nm.

32. The sensing substrate of any of claims 29-31, wherein the high-amplification regions have capture agents bound thereto.

33. The sensing substrate of any of claims 29-32, wherein the signal amplification layer is a D2PA.

34. The sensing substrate of any of claims 29-32, wherein the signal amplification layer comprises one or a plural of metallic discs and a significantly continuous metallic film, wherein the significant part of the metallic disc is separated from the metallic film.

35. The sensing substrate of claim 34, wherein the metallic disk has a shape selected from the group of shapes consisting of round, polygonal, pyramidal, elliptical, elongated bar shaped, or any combination thereof.

36. The sensing substrate of claim 34, wherein the metallic disc is separated from the metallic film by a distance in the range of 0.5 to 30 nm, and the average lateral dimension of the discs is in the range of 20 nm to 250 nm.

37. The sensing substrate of any of claims 29-32, wherein the signal amplification layer comprises one or more metallic discs has a shape selected from the group of shapes consisting of round, polygonal, pyramidal, elliptical, elongated bar shaped, or any combination thereof, wherein the average lateral dimension of the discs is in the range 20 nm to 250 nm, and the gap between adjacent discs in the range of 0.5 to 30 nm.
